(19) United States
(12) Patent Application Publication
(10) Pub. No.: US 2011/0294890 A1
(43) Pub. Date: Dec. 1, 2011

(54) Neramexane for the treatment or prevention of inner ear disorders

(75) Inventors: Michael Althaus, Schoeneck (DE); Barbara Ellers-Lenz, Moerfelden-Walldorf (DE); Irena Pulte, Eschborn (DE)

(73) Assignee: MERZ PHARMA GmbH & CO. KGaA, FRANKFURT am MAIN (DE)

(21) Appl. No.: 13/068,984

(22) Filed: May 25, 2011

Related U.S. Application Data
(60) Provisional application No. 61/349,223, filed on May 28, 2010.
(60) Provisional application No. 61/402,179, filed on Aug. 25, 2010.

(30) Foreign Application Priority Data
May 28, 2010 (EP) 100 05 584.7

(51) Int. Cl. A61K 31/13; A61P 27/16

(52) U.S. Cl. 514/579

(57) ABSTRACT

The present invention relates to a method of treatment or prevention of an inner ear disorder in a female comprising administering a therapeutically effective amount of neramexane or a pharmaceutically acceptable salt thereof to a female.

### FIELD OF THE INVENTION

The present invention relates to a method of treatment or prevention of an inner ear disorder in a female comprising administering a therapeutically effective amount of neramexane or a pharmaceutically acceptable salt thereof to a female.

### BACKGROUND OF THE INVENTION

Inner ear disorders are an increasing problem in nowadays society. The most common of these disorders, tinnitus is commonly referred to as ‘ringing in the ears’—the perception of sounds in the absence of an external source of acoustic signals. Tinnitus has been defined as “the perception of a sound which results exclusively from the activity within the nervous system without any corresponding mechanical, vibratory activity within the cochlea, that is, tinnitus as an auditory phantom perception” (Jastreboff et al., J Am Acad Audiol 2000; 11(3): 162-177). For the individual patient, tinnitus may be tolerable or it may represent a debilitating illness preventing its sufferers from sleep or work. Tinnitus is frequently associated with a decreased sound tolerance (i.e. hyperacusis).

The pathophysiology of subjective tinnitus is poorly understood and a definitive pathogenesis of tinnitus is unknown. Many environmental and substance-induced factors may cause tinnitus. Among the most frequently cited factors are acute acoustic trauma, occupational noise, and recreational music. In general, tinnitus seems to be the result of neuronal dysfunction within the auditory pathway. This dysfunction is misleadingly perceived as sound by higher auditory, centers and can lead to functional alterations within the auditory nervous system. Maladaptive functional changes in cortical structures could result in an altered balance between excitatory and inhibitory neurotransmission and may lead to more severe tinnitus. In all cases, a potential malfunction in auditory pathways and auditory cortex is related to the activity of the prefrontal cortex and limbic system.

In most cases (95%), the perceived tinnitus is purely subjective in nature, e.g. no physical source of acoustic signals can be identified and, therefore, cannot be heard externally. A physical examination is performed to exclude objective tinnitus, e.g. the patient's perception of sound is caused by a real source of sound waves, e.g. the sound from turbulent flow in blood vessels reaching the cochlea. Tinnitus may be classified according to duration of tinnitus and the degree of tinnitus expression (e.g. severity or annoyance of the tinnitus) (McCombe et al., Clin Otolaryngol 2001; 26(5): 388-393 and Davis et al., Epidemiology of Tinnitus. In: Tyler R, editor. Tinnitus Handbook. San Diego: Singular Publishing Group; 2000. p. 1-23). Regarding the impact of tinnitus, tinnitus may be severely annoying to the patient and can be accompanied by social and psychological complications.

There are currently no well-established, specific medical treatments for tinnitus that provide replicable reduction of tinnitus and annoyance due to tinnitus, in excess of placebo effects (Dobie, Laryngoscope 1999; 109(8): 1202-1211; Eggermont et al., Trends Neurosci 2004; 27(11): 676-682; and Patterson et al., Int Tinnitus J 2006; 12(2): 149-159).

Neramexane (also known as 1-amino-1,3,3,5,5-pentamethylcyclohexane) have been found to be useful in the therapy of various diseases especially in certain neurological diseases. Neramexane is disclosed, for example, in detail in U.S. Pat. Nos. 6,034,134 and 6,071,966. It is believed that the therapeutic action of neramexane is related to the inhibition of the effects of excessive glutamate at the N-methyl-D-aspartate (NMDA) receptors of nerve cells, for which reason the compounds are also categorized as n NMDA antagonists, or NMDA receptor antagonists. Neramexane has also been disclosed to exhibit activity at the α9/α10 nicotinic (Plazas, et al., Eur J Pharmacol., 2007 Jul. 2; 566(1-3):11-19) and 5-HT₃ receptors.

It was found earlier that neramexane may be useful in treating inner ear disorders such as tinnitus (WO 2009/033649, WO 2009/033651, WO 2009/033652, W0 2009/033650). During the course of continuing developing neramexane and its pharmaceutically acceptable salts thereof as a medicament for the prevention or treatment of inner ear disorders, it was revealed in phase III clinical trials that neramexane and its pharmaceutically acceptable salts, e.g. neramexane mesylate, are particularly effective in treating such disorders in female patients. The present invention is based on this finding.

### SUMMARY OF THE INVENTION

The present invention relates to Neramexane or a pharmaceutically acceptable salt thereof such as neramexane mesylate for the treatment or prevention of an inner ear disorder, wherein said neramexane or a pharmaceutically acceptable salt thereof is administered to a female.

In another embodiment it relates to the use of neramexane or a pharmaceutically acceptable salt thereof such as neramexane mesylate for the manufacture of a medicament for the treatment or prevention of an inner ear disorder, wherein said medicament is administered to a female.

In a further embodiment, the pharmaceutically acceptable salt of neramexane is neramexane mesylate.

In a still further embodiment, neramexane or a pharmaceutically acceptable salt thereof such as neramexane mesylate is administered in a body weight-adjusted target dose of 50 mg/day for females with up to 90 kg body weight or 75 mg/day for females with a body weight of ≧90 kg.

In a still further embodiment, the inner ear disorder is selected among tinnitus, vertigo, hearing loss, chronic ear pain, perilymphatic fistula, secondary endolymphatic hydrops, labyrinthitis and vestibular neuritis, acoustic neurome, otootxicity, autoimmune inner ear diseases (AIED) and Meniere's disease.

In a further embodiment, the inner ear disorder is selected among sub-acute tinnitus, cochlear tinnitus, paroxymal position vertigo (BPPV), acoustic trauma, noise-induced hearing loss, sensorineural hearing loss, mixed hearing loss, unspecified hearing loss, ototoxic hearing loss (ototoxicity), drug-induced hearing loss, environmental chemicals-induced hearing loss, cancer-induced hearing loss, surgical-induced hearing loss, radiation-induced hearing loss, infection-induced hearing loss, sudden (idiopathic) hearing loss, auditory processing disorder, and presbycusis.

In a still further embodiment, the inner ear disorder is sub-acute tinnitus.

In a still further embodiment, the inner ear disorder is subacute tinnitus and the pharmaceutically acceptable salt of neramexane is neramexane mesylate.

In a still further embodiment, neramexane or a pharmaceutically acceptable salt thereof such as neramexane mesylate is administered via a titration scheme that comprises the up-titration thereof.

In a further embodiment, said up-titration of neramexane or a pharmaceutically acceptable salt thereof such as neramexane mesylate is performed over a period of from four to five weeks to achieve an effective dose.

In a further embodiment the up-titration of neramexane or a pharmaceutically acceptable salt thereof such as neramexane mesylate is performed over a period of from four to five weeks to achieve an effective dose of from 5 to 150 mg per day.

In a further embodiment, said titration scheme comprises up-titration of neramexane or a pharmaceutically acceptable salt thereof such as neramexane mesylate in increasing dosages of 25 mg or 12.5 mg steps at weekly intervals.

In a further embodiment, the titration scheme comprises up-titration of neramexane, or a pharmaceutically acceptable salt thereof such as neramexane mesylate over a period of four weeks to achieve an effective dose of 50 mg or over a period of five weeks to achieve an effective dose of 75 mg per day.

In a further embodiment, neramexane or a pharmaceutically acceptable salt thereof such as neramexane mesylate is administered according to one of the following schedules:
once daily at a dose of 12.5 mg per day for the first week, twice daily, wherein each dose is 12.5 mg for the second week, twice daily, wherein one dose is 12.5 mg and the other dose is 25 mg for the third week, and twice daily, wherein each dose is 25 mg for the fourth week;
or
once daily at a dose of 12.5 mg per day for the first week, twice daily, wherein each dose is 12.5 mg for the second week, twice daily, wherein one dose is 12.5 mg and the other dose is 25 mg for the third week, and twice daily, wherein each dose is 25 mg for the fourth week, and twice daily, wherein each dose is 37.5 mg for the fifth week;
or
once daily at a dose of 25 mg per day for the first week, once daily at a dose of 50 mg per day for the second week, and, optionally, once daily at a dose of 75 mg per day for the third week.

In a further aspect of the invention neramexane mesylate is administered according to one of the above-recited schedules. If another pharmaceutically acceptable salt, a solvate, an isomer, a conjugate, a prodrug, a polymorphic form, or a derivative thereof, such as neramexane hydrochloride, is administered, equimolar amounts of another pharmaceutically acceptable salt, a solvate, an isomer, a conjugate, a prodrug, polymorphic form, or a derivative thereof, such as neramexane hydrochloride, may also be suitable.

Further aspects of the up-titration scheme recited above may be deduced from WO 2009/033651, which is incorporated herein by reference.

In a further embodiment, in weeks during which mixed doses are administered, the dose comprising the higher concentration is administered in the second daily dose.

In a further embodiment, the neramexane or a pharmaceutically acceptable salt thereof such as neramexane mesylate is administered once a day, twice a day (b.i.d.), or three times a day.

In a further embodiment, the neramexane or a pharmaceutically acceptable salt thereof such as neramexane mesylate is administered twice a day.

In a further embodiment, neramexane or a pharmaceutically acceptable salt thereof such as neramexane mesylate is administered in an immediate release formulation.

In a further embodiment, neramexane or a pharmaceutically acceptable salt thereof such as neramexane mesylate is administered in a modified release formulation.

In one embodiment of the present invention, the females are 26 to 65, or 46 to 65 years of age.

In another embodiment, the females without clinical signs of depression, in particular exhibited a rating of 7 or less according to the Hospital Anxiety and Depression Scale-Depression Subscale (HADS-D) Questionaire (Zigmond A S, Snaith R P: The Hospital Anxiety And Depression Scale, Acta Psychiatr Scand 1983, 67:361-70).

In a further embodiment the females to be treated are without clinical signs of depression, as defined above, and are 26 to 65 years of age.

In a still further embodiment the females are of the above defined age and/or are without clinical signs of depression, as defined above, and the inner ear disorder to be treated or prevented is tinnitus, e.g. sub-acute tinnitus.

In a further embodiment, the present invention relates to a method of treatment or prevention of an inner ear disorder in a female comprising administering a therapeutically effective amount of neramexane or a pharmaceutically acceptable salt thereof such as neramexane mesylate to a female.

In further embodiments said method of treatment or prevention can also be applied in accordance with the embodiments under [0010] to [0032].

### DETAILED DESCRIPTION OF THE INVENTION

As used herein, the term “inner ear disorder” includes, but is not limited to, tinnitus, vertigo, such as paroxymal position vertigo (BPPV), hearing loss inclusive subindications such as acoustic trauma, noise-induced hearing loss, sensorineural hearing loss, mixed hearing loss, unspecified hearing loss, ototoxic hearing loss (ototoxicity), drug-induced hearing loss, environmental chemicals-induced hearing loss, cancer-induced hearing loss, surgical-induced hearing loss, radiation-induced hearing loss, infection-induced hearing loss, sudden (idiopathic) hearing loss, auditory processing disorder, and presbycusis, perilymphatic fistula, secondary endolymphatic hydrops, labyrinthitis and vestibular neuritis, acoustic neuroma, autoimmune inner ear disease (AIED), chronic ear pain, and Meniere's disease.

As used herein, the term “tinnitus” includes, but is not limited to, all manifestations of subjective and objective tinnitus as well a acute, subacute and chronic forms. It also includes cochlear tinnitus as well as tinnitus associated with hearing loss or mild hearing loss.

As used herein the term “subacute tinnitus” includes tinnitus of a duration of three (3) to twelve (12) months, i.e. tinnitus of a duration of at least (equal or more) than three months up to less or equal to twelve months. Thus, the treatment thereof occurs within three to twelve months of onset of tinnitus, i.e. at least (equal or more) than three months up to less or equal to twelve months of onset of tinnitus. In another embodiment the treatment occurs within three to eight months of onset of tinnitus, i.e. at least (equal or more) than three months up to less or equal to eight months of onset of tinnitus.

As used herein, the term “cochlear tinnitus” refers to tinnitus in a frequency range of hearing loss. The term cochlear tinnitus includes motor tinnitus, cochlear motor tinnitus or hair cell tinnitus. Cochlear tinnitus may be caused by toxic drugs (e.g. loop diuretics such as frusemide, aminoglycosides such as gentamicin) or loud sound exposure (e.g. acoustic trauma, chronic occupational noise).

As used herein the term “hearing loss” (or “hearing impairment”) is a full or partial loss of the ability to detect sounds or to distinguish among different sounds. Hearing loss is diagnosed clinically by an increased hearing threshold level in a pure tone audiogram. The hearing threshold level of left/right ear in decibel (dB) may be calculated as the average of the actual numbers at different frequencies on a pure tone audiogram, ie. as the average hearing level threshold levels at 0.25, 0.5, 1, 2 and 4 kHz. There are different degrees of hearing loss. Hearing loss is defined herein as “mild hearing loss” (or “mild hearing impairment”) if the hearing threshold level is within 20-40 decibel (dB). Noise-induced tinnitus associated with hearing loss may be caused by acute or chronic conditions. Long-term exposure to excessive noise is the more common cause of noise-induced tinnitus associated with hearing loss; however, such tinnitus associated with hearing loss may also be caused by extremely loud sounds. Sensorineural tinnitus associated with hearing loss is due to insensitivity of the inner ear or to impairment of function in the auditory nervous system. Sensorineural tinnitus associated with hearing loss may be caused by abnormalities in the hair cells of the organ of the Corti in the cochlea.

Noise-induced hearing loss may be caused by acute or chronic conditions. Long-term exposure to excessive noise is the more common cause of noise-induced hearing loss; however, such hearing loss may also be caused by extremely loud sounds.

Sensorineural hearing loss is due to insensitivity of the inner ear or to impairment of function in the auditory nervous system. Sensorineural hearing loss may be caused by abnormalities in the hair cells of the organ of the Corti in the cochlea.

Ototoxic hearing loss may be caused by medications which damage the ear (i.e., drug-induced hearing loss). Such medications include chemotherapeutic (i.e., anti-neoplastics or anti-cancer) agents (such as cisplatin), aminoglycosides (such as gentamicin), diuretics (such as bumetanide), salicylates (such as aspirin), quinines, NSAIDS, and macrolide antibiotics.

Environmental chemicals-induced hearing loss may be caused by agents (i.e., environmental chemicals) which damage the ear (such as butyl nitrite, mercury or toluene).

Cancer-induced hearing loss may be caused by tumors in the middle ear as well as by other cancers which involve the ear and/or brain.

Surgical-induced hearing loss may occur after otologic or non-otologic surgery; however, the mechanism(s) associated with such hearing loss are not clear.

Radiation-induced hearing loss may be caused by intentional (for example, in radiation therapy) or unintentional exposure to radiation.

Infection-induced hearing loss may be caused by infections involving the inner ear and hearing nerve as well as by infections involving the middle ear. Moreover, there are a number of other types of infections (e.g., mumps, lyme disease, meningitis, herpesvirus infections, fungal infections, bacterial infections, AIDS, and tuberculosis) which may result in hearing loss.

Presbycusis appears to be related, in part, to noise exposure and is characterized by a stiffening of the basilar membrane and deterioration of the hair cells, stria vasularis, ganglion cells, and cochlear nuclei.

The terms “female”, “female patient” and “woman/women” as used herein encompasses female mammals including animals and humans. The term encompasses anybody who is of feminine sex or gender without any limitation as to age. In one embodiment the females to be treated are 18 years and older.

Neramexane is, among other 1-aminoalkylcyclohexanes, disclosed in U.S. Pat. Nos. 6,034,134 and 6,071,966. Neramexane (1-amino-1,3,3,5,5-pentamethylcyclohexane) may be used according to the invention also in the form of any of pharmaceutically acceptable salts, solvates, isomers, conjugates, prodrugs, polymorphic forms, derivatives, and mixtures thereof and any references to neramexane in this description should be understood as also referring also to such pharmaceutically acceptable salts, solvates, isomers, conjugates, prodrugs, polymorphic forms, derivatives, and mixtures thereof, unless explicitly indicated differently.

For the purpose of this disclosure, the term “pharmaceutically acceptable salt” denotes a salt form of 1-amino-1,3,3,5,5-pentamethylcyclohexane obtained by combination with an inorganic or organic acid which does not affect the safety of a human being and/or is well-tolerated by a human being after administration. Examples of pharmaceutically acceptable salts include, but are not limited to, acid addition salts, such as those made with hydrochloric, methylsulfonic, hydrobromic, hydroiodic, perchloric, sulfuric, nitric, phosphoric, acetic, propionic, glycolic, lactic, pyruvic, malonic, succinic, fumaric, tartaric, citric, benzoic, carbonic, cinnamic, mandelic, methanesulfonic, ethanesulfonic, hydroxyethanesulfonic, benzenesulfonic, p-toluene sulfonic, cyclohexanesulfamic, salicyclic, p-aminosalicylic, 2-phenoxybenzoic, and 2-acetoxybenzoic acid. All of these salts (or other similar salts) may be prepared by conventional means. The nature of the salt is not critical, provided that it is non-toxic and does not substantially interfere with the desired pharmacological activity. Conversion of 1-amino-1,3,3,5,5-pentamethylcyclohexane to a pharmaceutically acceptable salt may be accomplished in conventional fashion by admixture of the base with at least one molecular equivalent of a selected acid in an inert organic solvent. Isolation of the salt may be carried out by techniques known to the art such as inducing precipitation with a non-polar solvent (e.g. ether) in which the salt has limited solubility.

The term “pharmaceutically acceptable” in connection with an ingredient (or substance or compound or agent) encompasses an ingredient (or a substance or compound or agent) which does not affect the safety of a human being and/or is well-tolerated by a human being after administration. Typically, as used herein, the term “pharmaceutically acceptable” means approved by a regulatory agency or listed in a generally recognized pharmacopeia for use in mammals, and more particularly in humans.

The terms “polymorphic form” and “polymorphic forms” encompass neramexane or a pharmaceutically acceptable salt thereof forming different crystal structures or lattices.

The term “prodrug” encompasses a substance that is derived from neramexane or a substance from which neramexane may be prepared in vivo, and which is administered in an inactive or less active form compared to neramexane itself.

The term “solvate” encompasses a product, wherein 1-amino-1,3,3,5,5-pentamethylcyclohexane is associated with molecules of a solvent, or attracts such molecules. If the solvent is water, the solvate is also termed as “hydrate”.

The term “conjugate” encompasses a product, wherein neramexane is covalently or non-covalently attached to a carrier.

The term “derivative” encompasses neramexane wherein the amino group is derivatized with one or two alkyl groups.

The term “isomers” encompasses possible stereoisomers of neramexane such as conformational isomers and enantiomers or diastereomers.

The term “treat” is used herein to mean to relieve or alleviate at least one symptom of a disease or a condition in a subject. Within the meaning of the present invention, the term “treat” also denotes to arrest, delay the onset (i.e., the period prior to clinical manifestation of a disease) and/or reduce the risk of developing or worsening a disease.

The term “therapeutically effective” applied to dose or amount refers to that quantity of a compound or pharmaceutical composition that is sufficient to result in a desired activity upon administration to a mammal in need thereof.

The term “carrier” applied to pharmaceutical compositions of the invention refers to a diluent, excipient, or vehicle with which an active compound (e.g., neramexane) is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Such carriers can also be solids, for example excipients as described below in [0066]. Suitable pharmaceutical carriers are described in “Remington's Pharmaceutical Sciences” by A. R. Gennaro, 20^{th} Edition.

The term “about” or “approximately” usually means within 20%, alternatively within 10%, including within 5% of a given value or range.

The term “titration scheme” is meant to be a method of treatment as discussed herein, wherein patients are treated for a disease or a condition wherein at least two different dosages (doses) of neramexane or a pharmaceutically acceptable salt thereof, e.g. in the form of a pharmaceutical compositions useful in treating such condition are administered in a step-wise manner in a once daily or multiple times per day manner and wherein lower doses are administered earlier in the treatment and higher doses are administered during subsequent treatment weeks. Optionally, in those treatment weeks wherein different dosages are administered on the same day, the titration scheme may provide for the administration of a lower dosage in the morning and a higher dosage in the evening, thereby minimizing drug-induced side effects during the most productive hours of the day.

Neramexane or pharmaceutically acceptable salts thereof, such as neramexane mesylate, or a pharmaceutical composition comprising the same may be used for the treatment or prevention of an inner ear disorder in females according to the invention.

In one embodiment neramexane or a pharmaceutically acceptable salt thereof and/or pharmaceutical composition (medicament) are adapted to or appropriately prepared for a specific administration scheme as disclosed herein. For this purpose the package and/or the package leaflet and/or the patient information and/or the dosage form itself may contain corresponding information.

Neramexane or a pharmaceutically acceptable salt thereof, such as neramexane mesylate or the composition of the present invention may be used for the manufacture of a medicament for the treatment or prevention of an inner ear disorder, e.g. tinnitus, wherein the medicament is adapted to or appropriately prepared for a specific administration as disclosed herein. For this purpose the package leaflet and/or the patient information contains corresponding information.

According to the present invention, the dosage form of neramexane or a pharmaceutically acceptable salt thereof may be a solid formulation including a capsule, a tablet, or the like (see Remington's Pharmaceutical Sciences, 20^{th} Edition, by A. R. Gennaro).

Neramexane or a pharmaceutically, acceptable salt thereof may be administered orally as a semi-solid, or liquid formulation (see Remington's Pharmaceutical Sciences, 20^{th} Edition, by A. R. Gennaro).

For solid formulations in the form of a tablet or capsule, neramexane or a pharmaceutically acceptable salt thereof may be combined with non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like.

The tablets may be coated with a concentrated sugar solution which may contain e.g., gum arabic, gelatine, talcum, titanium dioxide, and the like. Alternatively, the tablets can be coated with a polymer that dissolves in a readily volatile organic solvent or mixture of organic solvents. In specific embodiments, neramexane is formulated in immediate-release (IR) or modified-release (MR) tablets. Immediate release solid dosage forms permit the release of most or all of the active ingredient (e.g. 90% or more) over a short period of time, such as 60 minutes or less, and make rapid absorption of the drug possible (immediate release formulations of 1-amino-alkylcyclohexanes such as neramexane are disclosed in US Published Application Nos. 2006/0002999 and 2006/0198884, the subject matter of which relating to immediate release formulations is hereby incorporated by reference). Modified release solid oral dosage forms permit the sustained release of the active ingredient over an extended period of time in an effort to maintain therapeutically effective plasma levels over similarly extended time intervals and/or to modify other pharmacokinetic properties of the active ingredient (modified release formulations of neramexane are disclosed in US Published Application No. 2007/0141148, the subject matter of which is hereby incorporated by reference). For example, neramexane mesylate may be formulated in a modified release dosage form (including modified release tablets) to provide a 50 mg dose of neramexane mesylate.

For the formulation of soft gelatin capsules, neramexane or a pharmaceutically acceptable salt thereof may be admixed with e.g., a vegetable oil or poly-ethylene glycol. Hard gelatin capsules may contain granules of the active substances using either the above mentioned excipients for tablets e.g., lactose, saccharose, sorbitol, mannitol, starches (e.g., potato starch, corn starch or amylopectin), cellulose derivatives or gelatine. Also liquids or semisolids of the drug can be filled into hard gelatine capsules.

Neramexane or a pharmaceutically acceptable salt thereof can also be introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publications No. WO 95/11010 and WO 93/07861). Biocompatible polymers may be used in achieving controlled release of a drug, include for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

Formulation of neramexane or a pharmaceutically acceptable salt thereof in a semi-solid or liquid form may also be used. Neramexane may constitute between 0.1 and 99% by weight of the formulation, more specifically between 0.2 and 50% by weight for formulations suitable for oral administration.

In one embodiment of the invention, neramexane or a pharmaceutically acceptable salt thereof is administered in a modified release formulation. Modified release dosage forms provide a means for improving patient compliance and for ensuring effective and safe therapy by reducing the incidence of adverse drug reactions. Compared to immediate release dosage forms, modified release dosage forms can be used to prolong pharmacologic action after administration, and to reduce variability in the plasma concentration of a drug throughout the dosage interval, thereby eliminating or reducing sharp peaks.

A modified release form dosage may comprise a core either coated with or containing a drug. The core being is then coated with a release modifying polymer within which the drug is dispersed. The release modifying polymer disintegrates gradually, releasing the drug over time. Thus, the outer-most layer of the composition effectively slows down and thereby regulates the diffusion of the drug across the coating layer when the composition is exposed to an aqueous environment, i.e. the gastrointestinal tract. The net rate of diffusion of the drug is mainly dependent on the ability of the gastric fluid to penetrate the coating layer or matrix and on the solubility of the drug itself.

In another embodiment of the invention, neramexane or a pharmaceutically acceptable salt thereof is formulated in an oral, liquid formulation. Liquid preparations for oral administration can take the form of, for example, solutions, syrups, emulsions or suspensions, or they can be presented as a dry product for reconstitution with water or other suitable vehicle before use. Preparations for oral administration can be suitably formulated to give controlled or postponed release of the active compound. Oral liquid formulations of 1-amino-alkylcyclohexanes, such as neramexane, are described in PCT International Application No. PCT/US2004/037026, the subject matter of which is hereby incorporated by reference.

For oral administration in liquid form, neramexane or a pharmaceutically acceptable salt thereof may be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms. For example, solutions may contain from about 0.2% to about 20% by weight of neramexane, with the balance being sugar and mixture of ethanol, water, glycerol and propylene glycol. Optionally, such liquid formulations may contain coloring agents, flavoring agents, saccharine and carboxymethyl-cellulose as a thickening agent or other excipients.

In another embodiment, a therapeutically effective amount of neramexane or a pharmaceutically acceptable salt thereof is administered in an oral solution containing a preservative, a sweetener, a solubilizer, and a solvent. The oral solution may include one or more buffers, flavorings, or additional excipients. In a further embodiment, a peppermint or other flavoring is added to the neramexane oral liquid formulation.

For administration by inhalation, neramexane may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Solutions for parenteral applications by injection may be prepared in an aqueous solution of a water-soluble pharmaceutically acceptable salt of the active substances, e.g., in a concentration of from about 0.5% to about 10% by weight. These solutions may also contain stabilizing agents and/or buffering agents and may conveniently be provided in various dosage unit ampoules.

The formulations of the invention may be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. Alternatively, the active ingredient may be in powder form for reconstitution with a suitable vehicle, e.g., sterile pyrogen-free water, before use.

The invention also provides a pharmaceutical pack or kit comprising one or more containers containing neramexane or a pharmaceutically acceptable salt thereof and, optionally, more of the ingredients of the formulation. In a specific embodiment, neramexane is provided as an oral solution (2 mg/ml) for administration with the use of a 2 teaspoon capacity syringe (dosage KORC®). Each oral syringe has blue hatch marks for measurement, with lines on the right side of the syringe (tip down) representing tsp units, and those on the left representing ml units.

The optimal therapeutically effective amount may be determined experimentally, taking into consideration the exact mode of administration in which the drug is administered, the indication toward which the administration is directed, the subject involved (e.g., body weight, health, age, sex, etc.), and the preference and experience of the physician or veterinarian in charge.

Dosage units for rectal application may be solutions or suspensions or may be prepared in the form of suppositories or retention enemas comprising neramexane in a mixture with a neutral fatty base, or gelatin rectal capsules comprising the active substances in admixture with vegetable oil or paraffin oil.

Toxicity and therapeutic efficacy of neramexane or a pharmaceutically acceptable salt thereof may be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it may be expressed as the ratio LD₅₀/ED₅₀. neramexane or a pharmaceutically acceptable salt thereof/compositions that exhibit large therapeutic indices are preferred.

Suitable daily doses of the active compounds of the invention in therapeutic treatment of humans are about 0.01-10 mg/kg bodyweight on peroral administration and 0.001-10 mg/kg bodyweight on parenteral administration. For example, for adults, suitable daily doses of neramexane mesylate include doses of 50 mg and 75 mg per day. An equimolar amount of another pharmaceutically acceptable salt, a solvate, an isomer, a conjugate, a prodrug, a polymorphic form, or a derivative thereof, such as neramexane hydrochloride, is also suitable.

In general neramexane or a pharmaceutically acceptable salt thereof such as neramexane mesylate is administered in a range from about 5 mg to about 150 mg/day, or in a range from about 5 mg to about 100 mg/day, or at about 5 mg to about 75 mg/day, or at about 50 mg/day, or at about 75 mg/day.

The daily doses indicated herein may be administered, for example, as one or two dosing units once, twice or three times per day. Suitable doses per dosage unit may therefore be the daily dose divided (for example, equally) between the number of dosage units administered per day, and will thus typically be about equal to the daily dose or one half, one third, one quarter or one sixth thereof. Dosages per dosage unit may thus be calculated from each daily dosage indicated herein. A daily dose of 5 mg, for example may be seen as providing a dose per dosage unit of, for example, about 5 mg, 2.5 mg, 1.67 mg, 1.25 mg and 0.83 mg, depending upon the dosing regimen chosen. Correspondingly, a dosage of 150 mg per day corresponds to dosages per dosing unit of, for example, about 150 mg, 75 mg, 50 mg, 37.5 mg, and 25 mg for corresponding dosing regimens.

Treatment duration may be short-term, e.g., several weeks (for example 8-14 weeks), or long-term until the attending physician deems further administration no longer is necessary.

A further aspect of the invention relates to the use of neramexane or a pharmaceutically acceptable salt thereof such as neramexane mesylate according to the invention, in combination with an additional pharmaceutical agent selected from antidepressants or anti-anxiety drugs (such as selective serotonin reuptake inhibitors (SSRIs), serotonin-norepinephrine reuptake inhibitors (SNRIs), noradrenergic and specific serotonergic antidepressants (NASSAs), norepinephrine (noradrenaline) reuptake inhibitors (NRIs), norepinephrine-dopamine reuptake inhibitors, or serotonin 1A agonists), dopamine modulators, Alpha2Delta ligands, and NK1 antagonists, and, optionally, at least one pharmaceutically acceptable carrier or excipient.

In this embodiment, neramexane or a pharmaceutically acceptable salt thereof such as neramexane mesylate and the additional pharmaceutical agent are administered conjointly or in a single formulation.

The term “combination” applied to active ingredients is used herein to define a single pharmaceutical composition (formulation) comprising two active agents (e.g., a pharmaceutical composition comprising neramexane, and another agent prescribed for the treatment of women or two separate pharmaceutical compositions, each comprising neramexane, or another agent prescribed for the treatment of women, to be administered conjointly.

Within the meaning of the present invention, the term “conjoint administration” is used to refer to administration of neramexane, and a second active agent (e.g. another agent prescribed for the treatment of women) simultaneously in one composition, or simultaneously in different compositions, or sequentially. For the sequential administration to be considered “conjoint”, however, neramexane, and the second active agent must be administered separated by a time interval which still permits the resultant beneficial effect for treating women according to the present invention.

### EXAMPLES

The following examples illustrate the invention without limiting its scope.

### Example 1

### Double Blind Placebo Controlled Phase III Clinical Trial of Neramexane for Treatment of Tinnitus

The objective of this clinical trial was to assess the efficacy of neramexane as a treatment for tinnitus. The primary objective of this 17 weeks double-blind, randomized placebo-controlled study was to compare the efficacy, tolerability and safety of neramexane mesylate at two different, weight-adapted dosages (50 or 75 mg/d) with placebo in subjects with subjective tinnitus of at least moderate severity.

### Study Design

In a double-blind, multicenter, randomized, placebo-controlled, parallel-group study, the efficacy of neramexane in subjects suffering from tinnitus of at least moderate severity was assessed. A total of 411 patients, who fulfilled particular inclusion criteria and met none of particular exclusion criteria, were randomized to one of two double-blind treatment groups (neramexane mesylate 50 or 75 mg/d depending on body weight, or placebo).

Subjects were treated for 17 weeks with neramexane or placebo including a four- resp. five-week up-titration period, depending on study drug dose, followed by 30 to 35 days treatment-free observational period. Alternatively to treatment-free observational period, subjects could immediately at Week 17 start with a one-year open label treatment study.

Main inclusion criteria were:
Diagnosis of “subjective tinnitus”
Tinnitus duration of at least 3 months and less or equal to 12 months (subacute tinnitus)
Persistent, uni- or bilateral tinnitus
THI-12≧9 (=at least moderate tinnitus severity)
Hospital Anxiety and Depression (HADS) subscores for depression ≦10 and for anxiety ≦10

Main exclusion criteria were:
Intermittent or pulsatile tinnitus
Tinnitus as a concomitant symptom of an otological/neurological disease
Concomitant tinnitus treatment
Application for a pension/retirement pay or granted pension/retirement because of tinnitus.

Patients with a target daily dose of 50 mg neramexane mesylate (<90 kg body weight) were to reach steady state after four weeks, patients with a target total daily dose of 75 mg neramexane mesylate (≧90 kg body weight) were to reach steady state after five weeks of treatment. For patients who experienced dose limiting adverse events with the 75 mg dose, the dosage was reduced by switching the patient to 50 mg/day. Patients unable to tolerate a minimum dosage of 50 mg/day discontinued.

The scheduled visits for evaluation of each patient were as follows: Blood pressure and vital signs were recorded at each visit as well as pregnancy test female patients with childbearing potential.

Visit 1 (Screening): After signing the consent form, the subject underwent a physical examination (including body weight), ECG, clinical laboratory testing, including a voluntary blood sampling for pharmacogenetic testing.

Patient eligibility for the study was evaluated via a check of inclusion/exclusion criteria. The subject completed a Tinnitus-Beeinträchtigungs-Fragebogen (TBF-12=Tinnitus. Handicap Inventory (THI-12)) (i.e., a 12-item modified and validated version (Greimel K V et al., Tinnitus-Beeinträchtigungs-Fragebogen (TBF-12). Manual. Frankfurt am Main: Swets & Zeitlinger B. V.; 2000) of the 25-item Tinnitus Handicap Inventory or THI (Newman C W, et al. Development of the Tinnitus Handicap Inventory. Arch Otolaryngol Head Neck Surg 1996; 122(2): 143-148; Newman C W, et al. Psychometric adequacy of the Tinnitus Handicap Inventory (THI) for evaluating treatment outcome. J Am Acad Audiol 1998; 9(2): 153-160.)). The TBF-12 items can be grouped in 2 factorial scores: an emotional-cognitive and a functional-communicational factor. At Visit 1. patients also completed a Tinnitus Rating Scale (TRS, Likert scores on Tinnitus Loudness, Tinnitus Impact on Life, and Tinnitus Annoyance), and the Hospital Anxiety and Depression Scale Audiology/psychoacoustic measurements were performed unless results from previous examinations performed no more than one year prior to Screening were available.

Visit 2 (Baseline): The subject was asked about adverse events and changes in concomitant medication/disease, which events/changes were documented. The subject was evaluated for study eligibility based on a review of the inclusion/exclusion criteria. The subject completed a TBF-12 and a TRS as well as an abridged version of the Sleep Questionnaire-B (SF-B) and a Quality of Life scale (SF-36). The subject was enrolled in the study and study medication (placebo or neramexane) was dispensed as described below.

Visit 3 (Week 2): The subject was asked about adverse events and changes in concomitant medication/disease, which events/changes were documented.

Visit 4 (Week 5): This visit occurred at the end of the 5-week up-titration sequence. The subject was asked about adverse events and changes in concomitant medication/disease, which events/changes were documented. Blood samples were collected in order to determine neramexane concentration. The subject completed a TBF-12, a TRS, and an abridged SF-B Questionnaire. Medication compliance was assessed, and medication for the next 4 weeks was dispensed as described below.

Visit 5 (Week 9): This visit occurred at the end of the first 4-week fixed-dose double-blind treatment period. The subject was asked about adverse events and changes in concomitant medication/disease, which changes are documented. Blood samples were collected in order to determine neramexane concentration. Medication compliance was assessed and medication for the next 4 weeks was dispensed as described below.

Visit 6 (Week 13): This visit occurred at the end of the second 4-week fixed-dose double-blind treatment period. The subject was asked about adverse events and changes in concomitant medication/disease, which changes are documented. The subject completed a TBF-12, a TRS, and an abridges SF-B Questionnaire. Medication compliance was assessed and, medication for the next 4 weeks was dispensed as described below.

Visit 7 (Week 17, end of treatment (EOT)). This visit occurred at the end of the 12-week fixed-dose double-blind treatment period. This visit was also to be performed for patients who discontinued prematurely. The subject was asked about adverse events and changes in concomitant medication/disease, which changes are documented. A clinical laboratory evaluation was performed as well as ECG and physical examination (including body weight). Blood samples were collected in order to determine neramexane concentration. The subject completed a TBF-12, a TRS, a HADS, an abridged SF-B, and a SF-36 Questionnaire. Medication compliance was assessed. Patients could start with the one year open-label study with neramexane.

Visit 8 (30 to 35 days after EOT): For patients who discontinued the study prematurely or who did not enter the one year open-label study: this visit occurred at the end of the 30-35 days follow-up period after EOT visit (Visit 7). Review of concomitant medications as well as the occurrence of adverse events since the last visit is conducted with subject.

### Administration of Neramexane

Neramexane mesylate immediate release tablets (12.5 mg and 25 mg, the manufacture thereof being disclosed in WO 2009/033649, whose respective content is incorporated herein by reference) and matching placebo tablets are administered as film coated tablets.

Medication was dispensed from Visit 3 to Visit 6. Study medication for each study day consisted of 2 separate tablets and 2 reserve tablets. The dosing schedule is shown in Table 1.

Throughout the double-blind treatment period, patients were to take 2×1 tablet of medication daily.

TABLE 1
Administration of Neramexane mesylate

| | | | | | | 12-week fixed-dose | 30-35 days |
|---|---|---|---|---|---|---|---|
| | 5-week double-blind up-titration period | | | | | double-blind period | follow-up |
| Treatment | Week | Week | Week | Week | Week | Weeks | Weeks |
| group | 1 | 2 | 3 | 4 | 5 | 6-17 | 18-22 |
| 75 mg/d dose | 0/12.5 | 12.5/12.5 | 12.5/25 | 25/25 | 37.5/37.5 | 37.5/37.5 (75 mg/d) | — |
| 50 mg/d dose | 0/12.5 | 12.5/12.5 | 12.5/25 | 25/25 | 25/25 | 25/25 (50 mg/d) | — |
| Placebo | 0/0 | 0/0 | 0/0 | 0/0 | 0/0 | 0/0 | — |

xx/xx refers to the morning/evening dose in mg, respectively

Following the up-titration period, the dose was to be kept stable until the end of the study. However, subjects experiencing dose-limiting adverse events at a daily dose of 75 mg could be reduced to 50 mg neramexane per day.

Subjects were instructed to take study medication twice a day, at the approximately time in the morning and in the evening. At the end of week 5, 9 13, and 17 (or upon early termination), patients returned to the study site bringing their blister boxes with them for an assessment of medication compliance.

For the purpose of this clinical trial, 555 persons were screened. Among those, a total of 411 patients were randomized (205 for Placebo treatment group and 206 for treatment with neramexane). 198 patients treated with placebo and 203 patients treated with neramexane were evaluable for efficacy analyses (Full Analysis Set (FAS)).

### Efficacy

Primary Outcome

The change in TBF-12 total score from baseline (Visit 2) to the endpoint visit (Visit 7, i.e. Week 17 or end of treatment) was the primary efficacy endpoint in this study.

Secondary Outcomes
TBF-12 and TBF-12 factorial scores (values and absolute change from baseline) at all post-baseline visits as well as responder rates
Sum score of Tinnitus loudness, Tinnitus annoyance and Tinnitus impact on life (Tinnitus Rating Scale, TRS).
Tinnitus loudness (11-point Likert scale).
Tinnitus annoyance (11-point Likert scale).
Tinnitus impact on life (11-point Likert scale)
Abridged SF-B
SF-36
HADS

### Data Analysis

All efficacy analyses were performed on the full analysis set (FAS) using the last-observation-carried-forward (LOCF) approach. For sensitivity purposes an analysis of the per-protocol set and of observed cases was performed additionally. All statistical tests used for testing the primary efficacy (confirmatory testing) and secondary efficacy criteria (exploratory), and all other statistical tests used for exploratory analyses were two-sided hypothesis tests performed at the 5% significance level. For all variables standard descriptive statistics were calculated.

Change from baseline (Visit 2) to Week 17 in TBF-12 total score was analyzed using an ANCOVA model with treatment group, gender and country as factors and baseline TBF-12 total score as covariate.

For secondary efficacy parameters, the comparison between neramexane and placebo was performed, if appropriate, by visit using an ANCOVA with treatment group, gender and country as factors and the corresponding baseline value of the efficacy parameter as covariate.

### DISCUSSION

This clinical study showed a statistically significant improvement for the treatment of tinnitus in women with neramexane compared to the effect found for the total patient population. This is evidenced by the TBF-12 total score values The respective results are shown in Table 2 below.

TABLE 2
Change in the TBF-12 total score from
baseline to Week 17/EOT (FAS-LOCF)

| | | Neramexane | Comparison |
|---|---|---|---|
| | Placebo | 50/75 mg | between treatment |
| | N | N | groups |
| Popu- | Mean change (SD) | Mean change (SD) | LSmean (ner − pla) |
| lation | LSmean | LSmean | P-value |
| Females | 66 | 74 | −1.7 |
| | −1.8 (4.6) | −3.1 (4.2) | 0.0122 |
| | −2.1 | −3.8 | |
| Total | 198 | 203 | −0.5 |
| | −2.2 (4.2) | −2.5 (4.3) | 0.1997 |
| | −2.5 | −3.0 | |

LSmean = Mean value, adjusted for covariates. LSmean (ner − pla) = estimated difference Neramexane − Placebo and p-value from ANCOVA, N = Number of Patients, SD = standard deviation

The clinical studies also showed an improvement for the treatment of tinnitus in woman, who are 26 to 65, or 46 to 65 of age, or who are free from clinical signs of depression as defined hereinbefore, or who fall within said age group and are free from said depression signs.

1. A method of treating or preventing an inner ear disorder in a female subject, including a human female subject, comprising administering a therapeutically effective amount of neramexane, or a pharmaceutically acceptable salt thereof, to the female subject.

2. The method of claim 1, wherein the pharmaceutically acceptable salt is neramexane mesylate.

3. The method of claim 1, wherein neramexane, or a pharmaceutically acceptable salt thereof, is administered in a body weight-adjusted target dose of 50 mg/day for females with up to 90 kg body weight or 75 mg/day for females with a body weight of ≧90 kg.

4. The method of claim 1, wherein the inner ear disorder is selected from tinnitus, vertigo, hearing loss, chronic ear pain, perilymphatic fistula, secondary endolymphatic hydrops, labyrinthitis and vestibular neuritis, acoustic neurome, otootxicity, autoimmune inner ear diseases (AIED) and Meniere's disease.

5. The method of claim 1, wherein the inner ear disorder is selected from sub-acute tinnitus, cochlear tinnitus, paroxymal position vertigo (BPPV), acoustic trauma, noise-induced hearing loss, sensorineural hearing loss, mixed hearing loss, unspecified hearing loss, ototoxic hearing loss (ototoxicity), drug-induced hearing loss, environmental chemicals-induced hearing loss, cancer-induced hearing loss, surgical-induced hearing loss, radiation-induced hearing loss, infection-induced hearing loss, sudden (idiopathic) hearing loss, auditory processing disorder, and presbycusis.

6. The method of claim 5, wherein the inner ear disorder is sub-acute tinnitus.

7. The method of claim 1, wherein the neramexane, or a pharmaceutically acceptable salt thereof, is administered via a titration scheme comprising the up-titration of the neramexane, or a pharmaceutically acceptable salt thereof.

8. The method of claim 7, wherein the up-titration is performed over a period of from four to five weeks.

9. The method of claim 7, wherein the titration scheme comprises up-titration of neramexane, or a pharmaceutically acceptable salt thereof, in increasing dosages of 25 mg or 12.5 mg steps at weekly intervals.

10. The method of claim 7, wherein the titration scheme comprises up-titration of neramexane, or a pharmaceutically acceptable salt thereof, over a period of four weeks to achieve an effective dose of 50 mg, or over a period of five weeks to achieve an effective dose of 75 mg per day.

11. The method of claim 7, wherein neramexane, or a pharmaceutically acceptable salt thereof, is administered according to one of the schedules as follows:
(a) once daily at a dose of 12.5 mg per day for the first week; then twice daily, wherein each dose is 12.5 mg for the second week; then twice daily, wherein one dose is 12.5 mg and the other dose is 25 mg for the third week; and twice daily, wherein each dose is 25 mg for the fourth week; or
(b) once daily at a dose of 12.5 mg per day for the first week; then twice daily, wherein each dose is 12.5 mg for the second week; then twice daily, wherein one dose is 12.5 mg and the other dose is 25 mg for the third week; then twice daily, wherein each dose is 25 mg for the fourth week; and twice daily, wherein each dose is 37.5 mg for the fifth week; or
(c) once daily at a dose of 25 mg per day for the first week; once daily at a dose of 50 mg per day for the second week; and, optionally, once daily at a dose of 75 mg per day for the third week.

12. The method of claim 11, wherein in weeks during which mixed doses are administered, the dose comprising the higher concentration is administered in the second daily dose.

13. The method of claim 1, wherein the neramexane, or a pharmaceutically acceptable salt thereof, is administered once a day, twice a day (b.i.d.), or three times a day.

14. The method of claim 1, wherein the neramexane, or a pharmaceutically acceptable salt thereof, is administered twice a day.

15. The method of claim 1, wherein the neramexane, or a pharmaceutically acceptable salt thereof, is administered in an immediate release formulation.

16. The method of claim 1, wherein the neramexane, or a pharmaceutically acceptable salt thereof, is administered in a modified release formulation.
